(19) United States
(12) Patent Application Publication
(10) Pub. No.: US 2019/0244710 A1
(43) Pub. Date: Aug. 8, 2019

(54) SYSTEM AND METHOD FOR CONTROLLING DATA MANAGEMENT SYSTEM

(71) Applicant: MATERNAL METRICS, INC., North Hollywood, CA (US)

(72) Inventors: Lisa M. Korst, North Hollywood, CA (US); Moshe Fridman, Los Angeles, CA (US)

(21) Appl. No.: 16/262,759

(22) Filed: Jan. 30, 2019

Related U.S. Application Data
(60) Provisional application No. 62/625,612, filed on Feb. 2, 2018.

(51) Int. Cl. G16H 50/30; G16H 10/60

(52) U.S. Cl. CPC G16H 50/30; G16H 10/60

(57) ABSTRACT

A system is configured to: receive, from an electronic device, antepartum patient data corresponding to a first patient from among a plurality of patients; compare the antepartum patient data with pre-stored aggregated antepartum patient data; generate risk factor data based on the comparison of the antepartum patient data with the pre-stored aggregated antepartum patient data; transmit, to the electronic device, a signal for displaying information corresponding to the risk factor data; receive, from the electronic device, postpartum patient data corresponding to the first patient; aggregate the postpartum patient data corresponding to the first patient with pre-stored aggregated postpartum patient data corresponding to one or more of the plurality of patients to generate updated aggregated postpartum patient data; calculate, based on the updated aggregated postpartum patient data, a performance metric value corresponding to a healthcare provider; and automatically transmitting a notification signal to a healthcare provider electronic device.

### CROSS-REFERENCE TO RELATED APPLICATION

The present application claims priority to and the benefit of U.S. Provisional Application Ser. No. 62/625,612, filed Feb. 2, 2018, the entire content of which is incorporated herein by reference.

### BACKGROUND

Patient-reported experiences and outcomes are an important component of health care quality assessment, and are being incorporated into systems of hospital performance measurement. The financial incentive of the federal Value-Based Purchasing Program, stipulating that Medicare reimbursement dollars be withheld from hospitals with poor patient satisfaction scores, creates a strong business case for childbirth hospitals to collect and utilize patient-reported data. With nearly four million births annually, childbirth is the number one reason for hospital admission, and women rely on the medical system to provide them with safe and appropriate care. Recently delivered women are important contributors to these scores. As measured through the Hospital Consumer Assessment of Healthcare Providers and Systems (HCAHPS) survey, patient satisfaction ratings of hospital services rely on the aggregate response from medical, surgical, and maternity care service lines. However, given the uniqueness of the maternity admission and the generic nature of the HCAHPS survey, HCAHPS results provide only minimal guidance to hospitals regarding direct approaches to improving their scores in the maternity service line.

Related-art systems may collect and store electronic patient health records and data, but there may not be any mechanism for automatically identifying and mitigating for risk factors that may lead to undesired health outcomes.

The above information disclosed in this Background section is only for enhancement of understanding of the background of the present disclosures, and therefore, it may contain information that does not form prior art.

### SUMMARY

One or more example embodiments of the present disclosure are directed to a system and method for managing a healthcare system.

One or more example embodiments of the present disclosure may further include systems, methods, and devices for assisting in the assessment and improvement of the hospital childbirth experience.

According to some example embodiments of the present invention, an analytical data computing system includes: a processor; and a memory coupled to the processor, the memory storing instructions which, when executed by the processor, cause the processor to: receive, from an electronic device, antepartum patient data corresponding to a patient from among a plurality of patients; compare the antepartum patient data with pre-stored aggregated antepartum patient data; generate risk factor data based on the comparison of the antepartum patient data with the pre-stored aggregated antepartum patient data; transmit, to the electronic device, a signal for displaying information corresponding to the risk factor data; receive, from the electronic device, postpartum patient data corresponding to the patient; aggregate the postpartum patient data corresponding to the patient with pre-stored aggregated postpartum patient data corresponding to one or more of the plurality of patients to generate updated aggregated postpartum patient data; calculate, based on the updated aggregated postpartum patient data, one or more performance metric values corresponding to a healthcare provider; and in response to the one or more performance metric values being below a threshold value, automatically transmit a notification signal to a healthcare provider electronic device, the notification signal comprising an alert message regarding the one or more performance metric values.

According to some example embodiments, the instructions further cause the processor to identify, based on the antepartum patient data and the pre-stored aggregated postpartum patient data, one or more mitigating factors for the risk factor data.

According to some example embodiments, the signal for displaying information corresponding to the risk factor data comprises information regarding the one or more mitigating factors.

According to some example embodiments, the instructions further cause the processor to identify, based on the updated aggregated postpartum data and the one or more performance metric values being below the threshold value, one or more mitigating factors for adjusting the one or more performance metric values.

According to some example embodiments, the notification signal comprises information regarding the one or more mitigating factors.

According to some example embodiments, the instructions further cause the processor to: receive one or more electronic medical records corresponding to the patient; and automatically generate the risk factor data based on the comparison of the antepartum patient data with the pre-stored aggregated antepartum patient data and the one or more electronic medical records.

According to some example embodiments, the instructions further cause the processor to calculate, based on the one or more performance metric values, a potential change in a reimbursement amount for a corresponding change in the one or more performance metric values.

According to some example embodiments of the present invention, in a method for controlling a data management system, the method includes: receiving, by a processor, from an electronic device, antepartum patient data corresponding to a patient from among a plurality of patients; comparing, by the processor, the antepartum patient data with pre-stored aggregated antepartum patient data; generating, by the processor, risk factor data based on the comparison of the antepartum patient data with the pre-stored aggregated antepartum patient data; transmitting, by the processor, to the electronic device, a signal for displaying information corresponding to the risk factor data; receiving, by the processor, from the electronic device, postpartum patient data corresponding to the patient; aggregating, by the processor, the postpartum patient data corresponding to the patient with pre-stored aggregated postpartum patient data corresponding to one or more of the plurality of patients to generate updated aggregated postpartum patient data; calculating, by the processor, based on the updated aggregated postpartum patient data, one or more performance metric values corresponding to a healthcare provider; and in response to the one or more performance metric values being below a threshold value, automatically transmitting, by the processor, a notification signal to a healthcare provider electronic device, the notification signal comprising an alert message regarding the one or more performance metric values.

According to some example embodiments, the method further includes identifying, by the processor, based on the antepartum patient data and the pre-stored aggregated postpartum patient data, one or more mitigating factors for the risk factor data.

According to some example embodiments, the signal for displaying information corresponding to the risk factor data comprises information regarding the one or more mitigating factors.

According to some example embodiments, the method further includes identifying, by the processor, based on the updated aggregated postpartum data and the one or more performance metric values being below the threshold value, one or more mitigating factors for adjusting the one or more performance metric values.

According to some example embodiments, the notification signal comprises information regarding the one or more mitigating factors.

According to some example embodiments, the method further includes receiving, by the processor, one or more electronic medical records corresponding to the patient; and automatically generating, by the processor, the risk factor data based on the comparison of the antepartum patient data with the pre-stored aggregated antepartum patient data and the one or more electronic medical records.

According to some example embodiments, the method further includes calculating, by the processor, based on the one or more performance metric values, a potential change in a reimbursement amount for a corresponding change in the one or more performance metric values.

According to some example embodiments of the present invention, an analytical data computing system includes: a processor; and a memory coupled to the processor, the memory for storing instructions which, when executed by the processor, cause the processor to: collect information describing an individual's antepartum values and preferences for childbirth services in a predetermined set of questionnaire items in a predetermined set of multiple childbirth experience domains; and generate, based on a predetermined set of risk factors that may prevent pregnant women from being satisfied with hospital childbirth services in these multiple domains, an output that identifies risk factors for being dissatisfied with hospital childbirth services for each individual patient; and provide feedback regarding these risk factors to patients and hospitals; and collect information describing an individual's actual childbirth and postpartum experience; and generate, based on a predictive model, an output that identifies those childbirth experience items or domains that most greatly impact childbirth hospital satisfaction scores; and compare participating hospitals to develop target goals for satisfaction rates in each of the childbirth experience domains of the predictive model; and identify, based on each individual hospital's patient data, those modifiable childbirth experience items or domains associated with the highest deficiency in childbirth satisfaction rates; and generate, based on a predictive model, an output that estimates potential revenue increases associated with the resolution of specific deficiencies; and provide feedback regarding deficiencies to individual hospitals; and repeat the above steps iteratively, in whole or in part, as hospitals develop interventions for improving the childbirth experience and use the above steps to guide them toward satisfaction score improvement.

According to some example embodiments, the output is a set of item scores for each patient (termed, “perceived vulnerability scores”), each score indicating (yes/no) a potential for being dissatisfied with a specific childbirth experience item. A value may also be assigned to each of the multiple childbirth experience domains, as an aggregate score for those domains that have multiple items.

According to some example embodiments, the instructions further cause the processor to generate an output that includes the de-identification and aggregation of these patient-perceived vulnerability scores across each participating hospital.

According to some example embodiments, the instructions further cause the processor to generate an output that includes the re-identification of specific patient-reported data for proactive hospital use in mitigating risk factors associated with patient dissatisfaction.

According to some example embodiments, the instructions further cause the processor to: collect information directly from an individual in the antepartum period for a predetermined set of risk factors for dissatisfaction with the childbirth experience; assign, based upon the information from the individual, a separate value to each of these risk factors; and generate, based on these values, a set of perceived vulnerability scores for each individual.

According to some example embodiments, the instructions further cause the processor to: provide an antepartum survey including a plurality of questions; receive response information from an individual for each of said plurality of questions of the survey; determine, from the information of each question, whether the response is indicative of one of a high versus low risk of dissatisfaction with the childbirth experience; assign values to a plurality of these risk factors (each assigned to a specific childbirth experiences questionnaire item or domain), an output suggestive of risk factors associated with childbirth dissatisfaction (perceived vulnerability scores).

According to some example embodiments, the instructions further cause the processor to generate a report of an individual patient's perceived vulnerability scores and educational material to the patient.

According to some example embodiments, the reports are presented by a computer.

According to some example embodiments, the educational material is provided substantially contemporaneously with the receiving of responses from the individual.

According to some example embodiments, the instructions further cause the processor to: collect a predetermined set of electronic medical records data from a participating hospital; determine, from the data provided, whether an individual patient has clinical risk factors suggestive of risk factors for childbirth dissatisfaction; assign values to a plurality of issues, an output suggestive of risk factors for childbirth dissatisfaction (clinical vulnerability scores).

According to some example embodiments, the instructions further cause the processor to generate a report of a hospital's aggregated and de-identified patient perceived and clinical vulnerability scores and educational material to the hospital administration.

According to some example embodiments, the instructions further cause the processor to generate a hospital report that includes the re-identification of specific patient-reported data for proactive hospital use in mitigating risk factors associated with patient dissatisfaction.

According to some example embodiments, the reports are provided on a routine/rolling basis.

According to some example embodiments, the instructions further cause the processor to generate an output that identifies a set of those issues/domains that most greatly impact childbirth hospital satisfaction scores.

According to some example embodiments, the instructions further cause the processor to: collect information directly from an individual during the postpartum period regarding the occurrence of a predetermined set of childbirth and postpartum experiences that includes satisfaction with multiple childbirth experience domains and a summary childbirth hospital satisfaction score; assign, based upon the information from the individual, and any information collected in the antepartum period, a separate value for each of these experiences; and generate, based on these values, an output that consists of a set of childbirth experience and satisfaction scores for each individual.

According to some example embodiments, the instructions further cause the processor to: analyze healthcare information utilizing bivariate analyses of predictor variables and dichotomized summary childbirth hospital satisfaction scores; and from this information, selecting highly associated variables to be eligible for the next step, which is: analyze healthcare information utilizing a predictive model represented by the equations z=β₁X₁+β₂X₂+β₃X₃+β₄X₄ . . . βₙ₋₁Xₙ₋₁+βₙ (significant and/or clinically important second-order terms)+C, and P_{high}_{_}_{satisfaction}=e^{z}/(1+e^{z}), wherein β₁, β₂, β₃, β₄, . . . βₙ₋₁ represent regression coefficients estimates, βₙ represents a vector of regression coefficient estimates for the potential second-order terms, X₁, X₂, X₃, X₄, . . . Xₙ₋₁ represent predictor variables, the values of the predictor variables are determined based upon the healthcare information.

According to some example embodiments, individuals with a P_{high}_{_}_{satisfaction} value higher than a threshold value are identified as individuals with high predicted satisfaction scores.

According to some example embodiments, for all hospitals in the aggregate, the predictor variables with statistically significant odds ratios for P_{high}_{_}_{satisfaction} are identified.

According to some example embodiments, the instructions further cause the processor to generate an output that identifies target goals for each dimension of the childbirth experience and compares hospitals regarding these target goals.

According to some example embodiments, the data are aggregated within participating hospitals, and a threshold value for a target goal is set.

According to some example embodiments, the data are assigned a value as meeting or not meeting the target goal.

According to some example embodiments, the analyzing steps cause the generation of reports that compare de-identified hospitals on a routine/rolling basis.

According to some example embodiments, the instructions further cause the processor to generate an output wherein those satisfaction dimensions that have rates that are farthest from target goals are identified and quantified in an individual hospital report on a routine/rolling basis.

According to some example embodiments, the instructions further cause the processor to generate an output that estimates potential revenue increases associated with deficiency resolution.

According to some example embodiments, the predictor variables have a defined quantitative relationship with P_{high}_{_}_{satisfaction}, and the change in any one of these predictor variables for a specific hospital may predict a quantifiable change in P_{high}_{_}_{satisfaction}.

According to some example embodiments, an estimated change in P_{high}_{_}_{satisfaction} can be used to predict an estimated change in federal reimbursement at the hospital level.

According to some example embodiments, the analyzing steps cause the generation of individual hospital reports that identify the dimensions of childbirth hospital satisfaction that are the highest priorities to remediate, and the financial incentives involved in doing so.

According to some example embodiments, the reports are generated on a routine/rolling basis.

According to some example embodiments, the instructions further cause the processor to iteratively use of data from all steps to track and improve the childbirth experience.

According to some example embodiments of the present invention, a computer readable storage device includes executable instructions that, when executed by a processor, cause the processor to assist with the generation of the following outputs: individual and hospital aggregate vulnerability score generation and reports; individual and hospital aggregate patient satisfaction scores across multiple childbirth hospital experience items and domains; predicted high satisfaction scores across all hospitals; specific deficiencies in hospital childbirth satisfaction items and domains; estimates of potential hospital revenue increases for the remediation of childbirth satisfaction deficiencies; and hospital comparison, tracking, and specific reports regarding childbirth satisfaction across multiple childbirth experience items and domains.

According to some example embodiments, feedback is provided to patients and hospitals regarding risk factors for childbirth hospital dissatisfaction or actual deficiencies in childbirth hospital satisfaction, across multiple childbirth experience items and domains.

### BRIEF DESCRIPTION OF THE DRAWINGS

A more complete appreciation of the present disclosure, and many of the attendant features and aspects thereof, will become more readily apparent as the disclosure becomes better understood by reference to the following detailed description when considered in conjunction with the accompanying drawings in which like reference symbols indicate like components, wherein:

FIG. 1 is a block diagram of a computing device according to some example embodiments;

FIG. 2 is a block diagram illustrating further details of a computing device according to some example embodiments;

FIG. 3 is a diagram of a distributed computing system according to some example embodiments; and

FIG. 4 is a flowchart illustrating an example method for determining patient vulnerability scores and areas of hospital deficiencies in childbirth experience domains, and providing feedback to patients and hospitals according to some example embodiments.

### DETAILED DESCRIPTION

Hereinafter, example embodiments will be described in more detail with reference to the accompanying drawings, in which like reference numbers refer to like elements throughout. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of” and “at least one selected from,” when preceding a list of elements, modify the entire list of elements and do not modify the individual elements of the list.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively.

The electronic or electric devices and/or any other relevant devices or components according to embodiments of the present disclosure described herein, such as a processor, neural networks, neural network based controllers, a motor, actuators, and various sensors may be implemented utilizing any suitable hardware, firmware (e.g. an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the example embodiments of the present disclosure.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

The present disclosure provides non-limiting examples of systems, methods, and devices for assisting in the assessment and improvement of the hospital childbirth experience. An optimal childbirth experience is obtained by matching a pregnant patient's clinical needs, values and preferences for childbirth services with the actual hospital childbirth services received in a predetermined set of multiple childbirth experience domains. Prior to giving birth (antepartum), consumer data, including but not limited to patient reports and electronic medical record data, may be received regarding pregnant women who are identified as planning to deliver at a specific hospital. These data are used to identify risk factors that may prevent pregnant women from being satisfied with hospital childbirth services. A set of scores (termed “vulnerability scores”) may be determined based on these data, and feedback may be provided to the patients and hospitals. After delivery (postpartum), consumer data may be received regarding the childbirth experience. Based on these antepartum and postpartum data, and by use of a predictive model, the system may identify those childbirth experience domains that most impact summary scores for women's satisfaction with hospital childbirth services. Finally, aggregate scores may be calculated and compared across participating hospitals to arrive at ideal, “target” scores for satisfaction with the multiple childbirth experience domains, and hospitals may be identified as meeting or not meeting the target scores. In childbirth experience domains where hospitals do not meet target scores, the system will estimate the potential hospital revenue increases that may occur if childbirth satisfaction deficiencies are remediated. Reports of reassessments of consumer antepartum and postpartum scores, and the financial impact over time, may provide iterative feedback and opportunity for ongoing improvement of the childbirth experience.

Some example embodiments of the present invention may include a system that assists childbirth hospitals to more effectively assess and improve their patients' childbirth experience, enabling hospitals to develop strategies to improve their patients' satisfaction and increase their revenue.

It is with respect to these and other general considerations that embodiments disclosed herein have been made. Also, although relatively specific problems may be discussed, it should be understood that the embodiments should not be limited to solving the specific issues described above or elsewhere in this disclosure.

Related art systems may collect and receive patient medical records and health data, and store such information for future access by health care providers. But such systems, while perhaps being efficient for storing data, do not provide any mechanism for health care providers and patients to be alerted or notified about risk factors for undesired patient outcomes.

According to some example embodiments of the present invention, antepartum patient data may be collected for individual patients. The antepartum patient data may include, for example, patient healthcare records, conditions, and vital data, as well as desired clinical outcomes for a medical procedure or delivery. Based on the antepartum patient data, and aggregated antepartum and postpartum data for a plurality of patients, the analytical data computing system may identify risk factors or vulnerabilities that the patient may have an actual outcome that would fall short of a satisfactory or desired outcome. Based on the identified risk factors, the analytical data computing system may then automatically generate a notification or report of various mechanisms that can be utilized or actions that can be taken in order to mitigate any risk factors to reduce the likelihood of a less-than-desirable clinical outcome for the patient. Accordingly, in contrast to related-art technical systems, embodiments of the present invention may enable healthcare providers and patients to be automatically alerted about potential risk factors for undesirable outcomes, as well as potential mitigating factors or actions that may reduce the likelihood of undesired outcomes. Thus, example embodiments may improve upon related-art technical patient data and healthcare management systems by providing a mechanism for early interventions and preventative actions to improve healthcare outcomes for patients.

Although some example embodiments of the present invention are described in antepartum and postpartum contexts (i.e., obstetrical or gynecological healthcare) embodiments of the present invention are not limited thereto. For example, according to some example embodiments, rather than collecting and analyzing antepartum and postpartum data about a patient as it relates to a pregnancy or childbirth, the analytical data computing system may collect and analyze data about a patient prior to and after any other suitable medical procedure, such as a surgery, medication protocol, physical therapy, and the like.

Additionally, according to some example embodiments, the analytical data computing system may collect data corresponding to clinical outcomes for a plurality of patients and aggregate the data. Based on the aggregated data, the analytical data computing system may then automatically generate a signal or notification for a healthcare electronic device regarding activities or factors that can be modified in order to improve patient outcomes or satisfaction. Thus, healthcare providers may be provided with a mechanism for automatically identifying areas of deficiencies and taking corrective action to improve patient outcomes.

In general, some example embodiments may enable the assessment and scoring of women's antepartum, intrapartum, and postpartum experiences in multiple predetermined childbirth experience and clinical domains, the determination of deficiencies in hospital satisfaction rates in these various domains, and the estimation of federal and other revenue increases that may occur if these deficiencies are remediated. One step of the method may include collecting information from an individual for a predetermined set of domains and related items using, for example, an antepartum survey questionnaire. Another step of the method may include generating an output that identifies risk factors for pregnant women's dissatisfaction with hospital childbirth services, and providing feedback about these risk factors to both patients and hospitals for possible proactive intervention. Another step of the method may include collecting information describing an individual's actual childbirth and postpartum experience, and generating, based on a predictive model, an output that identifies those childbirth experience domains/items that most greatly impact childbirth hospital satisfaction scores. Another step of the method may include developing target goals for satisfaction rates in each of the childbirth experience domains, and identifying those domains with the highest deficiency in satisfaction rates and the greatest potential for receiving increased federal revenue if these deficiencies are remediated. Cycling through these steps iteratively as hospitals develop strategies for addressing deficiencies may lead to improvement in the childbirth experience.

Pursuant to another embodiment of the present invention, there is provided a healthcare management system including a processor, and a memory operably coupled to the processor. The memory includes instructions that, when executed by the processor, may cause the processor to assign, based upon information from an individual, a separate antepartum value to each predetermined childbirth experience domain. A similar process occurs when assigning a separate postpartum value to each predetermined childbirth experience domain. The instructions of the memory, when executed by the processor, may further cause the processor to generate, based upon a predetermined predictive model, a predicted probability of childbirth satisfaction for each individual. Furthermore, the instructions of the memory may further cause the processor to generate target scores for each childbirth experience domain, and to identify, for each hospital, those domains in need of remediation and the potential for receiving increased federal revenue upon such remediation.

Pursuant to another embodiment of the present invention, there is provided a computer readable medium for a healthcare management system. The computer readable medium including instructions that, when executed by the healthcare management system, may cause the healthcare management system to assign, based upon information from an individual, a separate antepartum value to each predetermined childbirth experience domain. A similar process occurs when assigning a separate postpartum value to each predetermined childbirth experience domain. The instructions, when executed by the healthcare management system, may further cause the healthcare management system to generate, based upon a predetermined predictive model, a predicted probability of childbirth satisfaction for each individual. Furthermore, the instructions, when executed by the healthcare management system, may further cause the processor to generate target scores for each childbirth experience domain, and to identify, for each hospital, those domains in need of remediation and the potential for receiving increased federal revenue upon such remediation.

Thus, some example embodiments may provide a new and useful healthcare management system and method.

Some example embodiments may further provide a healthcare management system and method for identifying individual users at high risk of dissatisfaction with hospital childbirth services and providing feedback to both individuals and hospitals to enable proactive management.

Some example embodiments may further provide a computer readable medium for configuring a healthcare management system to identify individuals at high risk for dissatisfaction with hospital childbirth services and provide feedback to both individuals and hospitals.

Some example embodiments may further provide a healthcare management system and method to identify areas of patient experience deficiencies within hospitals and estimate the financial benefit that would accrue to hospitals if these deficiencies were resolved.

Some example embodiments may further include identifying patients at risk for a poor childbirth experience, and through this knowledge, assisting individual hospitals in the identification and implementation of antepartum, intrapartum, and postpartum strategies for improving their patients' hospital satisfaction scores, and thereby increasing revenue.

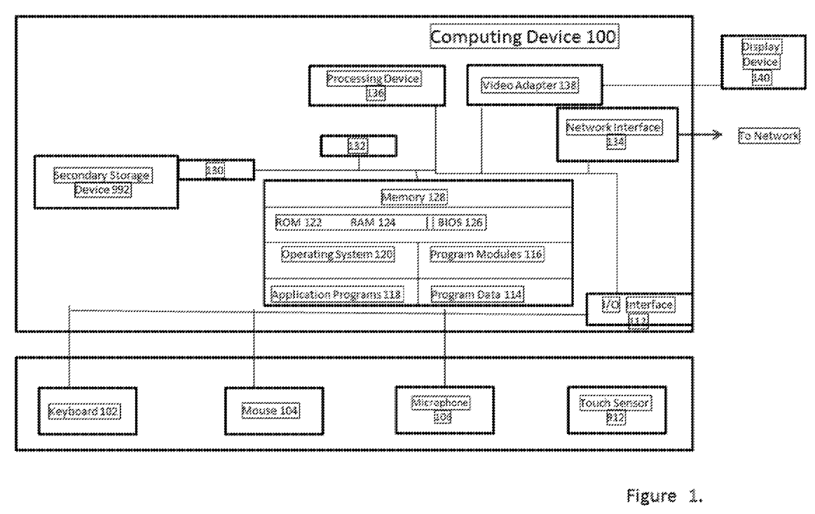

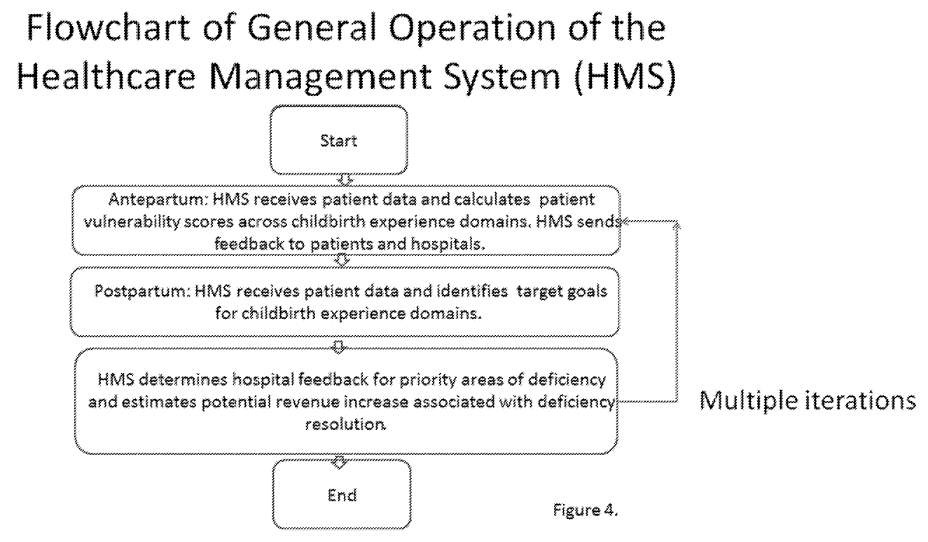

FIG. 1 illustrates one aspect in which an example architecture of a computing device according to the disclosure that can be used to implement aspects of the present invention, including any of the plurality of computing devices described herein with reference to the various figures. The computing device illustrated in FIG. 1 can be used to execute the operating system, application programs, and software modules (including the software engines) described herein, for example, with respect to FIG. 4. To avoid undue repetition, this description of the computing device will not be separately repeated herein for each of the other computing devices described herein and shown in the accompanying figures.

The computing device 100 includes, in some embodiments, at least one processing device 136, such as a central processing unit (CPU). A variety of processing devices are available from a variety of manufacturers, for example, Intel or Advanced Micro Devices. In this example, the computing device 100 also includes a system memory 128, and a system bus 132 that couples various system components including the system memory 128 to the processing device 136. The system bus 132 is one of any number of types of bus structures including a memory bus, or memory controller; a peripheral bus; and a local bus using any of a variety of bus architectures.

Examples of computing devices suitable for the computing device 100 include a server computer, a desktop computer, a laptop computer, a tablet computer, a mobile computing device (such as a smart phone, an iPod® or iPad® mobile digital device, or other mobile devices), or other devices configured to process digital instructions.

The system memory 128 includes read only memory 122 and random access memory 124. A basic input/output system 126 containing the basic routines that act to transfer information within computing device 100, such as during start up, is typically stored in the read only memory 122.

The computing device 100 also includes a secondary storage device 142 in some embodiments, such as a hard disk drive, for storing digital data. The secondary storage device 142 is connected to the system bus 132 by a secondary storage interface 130. The secondary storage devices 142 and their associated computer readable media provide nonvolatile storage of computer readable instructions (including application programs and program modules), data structures, and other data for the computing device 100.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 142 or memory 128, including an operating system 120, one or more application programs 118, other program modules 116 (such as the software engines described herein), and program data 114. The computing device 100 can utilize a suitable operating system, such as Microsoft Windows™, Google Chrome™, Apple OS, and any other operating system suitable for a computing device.

In some embodiments, a user provides inputs to the computing device 100 through one or more input devices 110. Examples of input devices 110 include a keyboard 102, mouse 104, microphone 106, and touch sensor 108 (such as a touchpad or touch sensitive display). Other embodiments include other input devices 110. The input devices are often connected to the processing device 136 through an input/output interface 112 that is coupled to the system bus 132. These input devices 110 can be connected by any number of input/output interfaces, such as a parallel port, serial port, game port, or a universal serial bus. Wireless communication between input devices and the interface 112 is possible as well, and includes infrared, BLUETOOTH® wireless technology, cellular and other radio frequency communication systems in some possible embodiments.

In this example embodiment, a display device 140, such as a monitor, liquid crystal display device, projector, or touch sensitive display device, is also connected to the system bus 132 via an interface, such as a video adapter 138. In addition to the display device 140, the computing device 100 can include various other peripheral devices (not shown), such as speakers or a printer.

When used in a local area networking environment or a wide area networking environment (such as the Internet), the computing device 100 is typically connected to the network through a network interface 134, such as an Ethernet interface. Other possible embodiments use other communication devices. For example, some embodiments of the computing device 100 include a modem for communicating across the network.

The computing device 100 typically includes at least some form of computer readable media. Computer readable media include any available media that can be accessed by the computing device 100. By way of example, computer readable media include computer readable storage media and computer readable communication media.

Computer readable storage media include volatile and nonvolatile, removable and non-removable media implemented in any device configured to store information such as computer readable instructions, data structures, program modules or other data. Computer readable storage media include, but are not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 100. Computer readable storage media do not include computer readable communication media.

Computer readable communication media typically embody computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media include wired media such as a wired network or direct-wired connection, and wireless media such as acoustic, radio frequency, infrared, and other wireless media. Combinations of any of the above are also included within the scope of computer readable media.

The computing device illustrated in FIG. 1 is also an example of programmable electronics, which may include one or more such computing devices, and when multiple computing devices are included, such computing devices can be coupled together with a suitable data communication network so as to collectively perform the various functions, methods, or operations discussed herein.

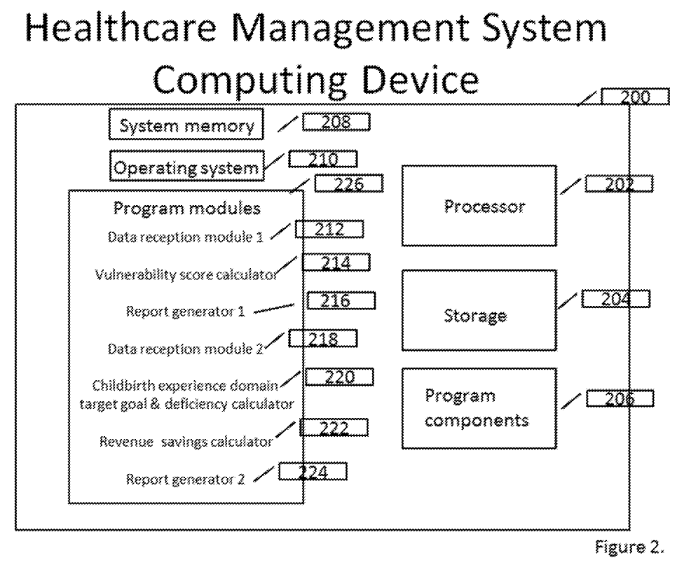

FIG. 2 is a block diagram illustrating additional physical components (e.g., hardware) of a computing device 200 with which certain aspects of the disclosure may be practiced. The computing device components described below may have computer executable instructions for receiving antenatal electronic medical record and consumer data; calculating patient vulnerability scores; providing feedback to patients and hospitals; receiving postpartum electronic medical record and consumer data; calculating childbirth experience domain target goals and hospital deficiencies; estimating potential revenue savings; and providing hospital feedback regarding goals and deficiencies.

Computing device 200 may perform these functions alone or in combination with a distributed computing network, which may be in operative contact with a personal computing device, a tablet computing device and/or mobile computing device which may communicate and process one or more of the program modules described in FIG. 2 including data reception module 1 212, vulnerability score calculator 214, report generator 1 216, data reception module 2 218, childbirth experience domain target goal & deficiency calculator 220, revenue savings calculator 222, and report generator 2 224.

In a basic configuration, the computing device 200 may include at least one processor 202 and a system memory 208. Depending on the configuration and type of computing device, the system memory 208 may comprise, but is not limited to, volatile storage (e.g., random access memory), non-volatile storage (e.g., read-only memory), flash memory, or any combination of such memories. The system memory 208 may include an operating system 212 and one or more program modules 226 suitable for performing the functions described herein with regard to vulnerability score generation and analysis and data communication, such as one or more components in regard to FIG. 2 and, in particular, data reception module 1 212, vulnerability score calculator 214, report generator 1 216, data reception module 2 218, childbirth experience domain target goals & deficiencies calculator 220, revenue savings calculator 222, and report generator 2 224. The operating system 210, for example, may be suitable for controlling the operation of the computing device 200. Furthermore, embodiments of the disclosure may be practiced in conjunction with a graphics library, other operating systems, or any other application program and are not limited to any particular application or system.

The computing device 200 may have additional features or functionality. For example, the computing device 200 may also include additional data storage devices (removable and/or non-removable) such as, for example, magnetic disks, optical disks, or tape. Such additional storage is illustrated in FIG. 2 by storage 204. Storage may also occur via distributed computing networks.

As stated above, a number of program modules and data files may be stored in the system memory 208. While executing the processor 202, the program modules 226 (e.g., data reception module 1) may perform processes including, but not limited to, the aspects described herein. Other program modules may be used in accordance with aspects of the present disclosure.

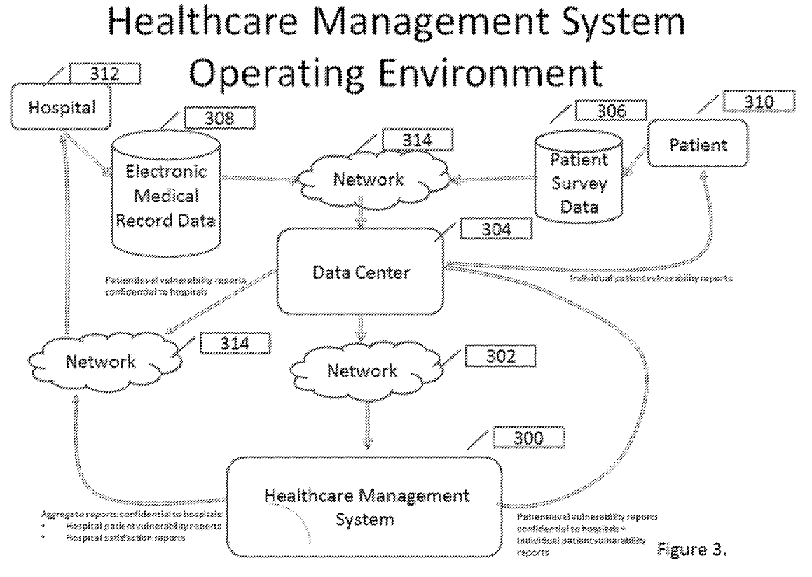

FIG. 3 is a simplified diagram of a distributed computing system in which aspects of the present disclosure may be practiced. In it, HMS 300 is connected to a network 302 that receives data from a third-party data center 304 that is contracted to collect, link, and de-identify patient data. Data may be solicited and collected through multiple classes of devices or networks 314, and include, but are not limited to patient survey data 306 and electronic medical record data 308. Upon processing these data, the HMS 300 may provide aggregated feedback to hospitals or other medical facilities 312 (de-identified at the patient level), or provide patient-level feedback to these facilities or patients. The HMS 300 does not directly receive identifiable patient data; therefore, individual-level data reports that either identify and report to hospitals regarding specific patients with high vulnerability scores or provide educational material to specific patients must be routed through the data center.

As stated above, the present disclosure provides non-limiting examples of systems, methods, and devices for assisting in the assessment and improvement of the hospital childbirth experience. An optimal childbirth experience is obtained by matching a pregnant patient's clinical needs, values and preferences for childbirth services with the actual hospital childbirth services received in a predetermined set of multiple childbirth experience domains. An example of the operations of the preferred embodiment of the HMS 300 is described below.

FIG. 4 is a flowchart depicting an example method for determining patient vulnerability scores and areas of hospital deficiencies in childbirth experience domains, and providing feedback to patients and hospitals.

To assure confidentiality, a third-party data center may receive antepartum patient data over the networks from participating hospitals or other medical organizations for a plurality of pregnant patients who are planning to deliver at a related facility. These data may be sent using a variety of devices or networks, and may include, but are not limited to, electronic medical record data and patient-reported data for a predetermined set of variables, including demographic information and a set of desired childbirth services in multiple childbirth experience domains. If data are received from multiple sources, they may be linked at the individual level by the data center and de-identified prior to exporting them over the Internet to the HMS 400 where they are received by Data Reception Module 1 212.

One example data source may be a survey where patients respond to a predetermined set of questionnaire items that comprise a predetermined set of childbirth experience domains. Each of these domains may have one item or multiple items.

Upon receipt of the antepartum data, the Vulnerability Score Calculator assigns a value to each of the multiple childbirth experience items, indicating whether the patient may have values or preferences that may conflict with the actual childbirth services she receives, and subsequently place her at risk for dissatisfaction with her childbirth experience. The Vulnerability Score Calculator may also assign a value to each of the multiple childbirth experience domains, as an aggregate score for those domains that have multiple items. For example, if, in the domain of “pain management” there are multiple similar items regarding the patient's desire for various pain management options, the scores assigned to each item in the domain may be averaged. Other examples of patient preferences that may not be fulfilled are: 1) provision of adequate food and space for her husband/partner; 2) ability to attempt a trial of labor to achieve a vaginal birth after having had a prior cesarean birth; 3) ability to stay in the hospital more than 2 days postpartum; and 4) ability to breastfeed the newborn. All of these preferences are risk factors for dissatisfaction because they represent problematic areas where pregnant women may have expectations that differ from routine practices in their chosen facility. Furthermore, they all represent services that are potentially mutable, i.e., services that hospitals may or may not choose to provide. Patients may use this information to clarify the availability of these services and seek hospitals that better meet their preferences. Hospitals may also benefit from knowledge regarding their patients that may be expecting services that they cannot provide. In Report Generator 1, specific items or domains where a patient has been identified as being at risk may be linked to educational material that is aggregated to form a report that is sent to the data center to be forwarded to the patient. Individual patient antepartum data may also be aggregated at the hospital level. A confidential report that summarizes the proportion of patients who are at risk in each of the multiple childbirth experience domains may be sent to the hospital to allow the potential for proactive management or policy development. Even more specifically, hospitals may request that the report identify the individuals who are at risk, and this report can be constructed by the HMS and returned to the data center to add patient identifiers.

The data center may be aware of the anticipated delivery dates for patients in the participating hospitals and may contact patients regarding their delivery experience during the postpartum period. In addition, hospitals may be contacted for electronic medical record data pertaining to the delivery. These data may be received by Data Reception Module 2 and linked to data in the Data Reception Module 1 if appropriate. Patients who did not provide data for Data Reception Module 1, but who are identified by the hospital after delivery, may be eligible for participation beginning with Data Reception Module 2 and may have a separate set of predetermined variables to be collected.

Data received by Data Reception Module 2 are analyzed by the Childbirth Experience Domain Target Goals and Deficiency Calculator. This calculator has several functions. First, data are received and scores may be assigned regarding a patient's satisfaction with childbirth services and whether she received the anticipated services. The calculator analyzes this information utilizing bivariate analyses of predictor variables and dichotomized summary childbirth hospital satisfaction scores. From this information, the calculator selects highly associated variables to be eligible for the next step, which is to analyze healthcare information utilizing a predictive model represented by the equations z=β₁X₁+β₂X₂+β₃X₃+β₄X₄ . . . βₙ₋₁Xₙ₋₁+βₙ (significant and/or clinically important second-order terms)+C, and P_{high}_{_}_{satisfaction}=e^{z}/(1+e^{z}), wherein β₁, β₂, β₃, β₄, . . . βₙ₋₁ represent regression coefficients estimates, βₙ represents a vector of regression coefficient estimates for the potential second-order terms, X₁, X₂, X₃, X₄, . . . Xₙ₋₁ represent predictor variables; the values of the predictor variables are determined based upon the healthcare information. For all hospitals in the aggregate, the predictor variables with statistically significant odds ratios for P_{high}_{_}_{satisfaction} are identified.

The calculator then determines target goals for each of the childbirth experience domain scores (e.g., identifies the threshold of the top quartile of the distribution of domain scores among participating hospitals), and scores hospitals regarding whether they did or did not meet the target. For hospitals that did not meet the target, the deficiency from the target is calculated. The next step is for the calculator to estimate the revenue that could be gained by remediation of these deficiencies. The relationship between HCAHPS hospital satisfaction scores and the amount of federal reimbursement withholding is published annually by the Centers for Medicare and Medicaid (CMS). The general relationship between the most important childbirth experience domain deficiencies and HCAHPS hospital satisfaction scores is calculated in the predictive model. The hospital-specific relationship between an individual hospital's scores in the most important childbirth experience domains and the HCAHPS hospital satisfaction scores can be calculated using the hospital's data in this model. This information can then be used to estimate the relationship between the amount of federal reimbursement withholding and the most important childbirth experience domain deficiencies, i.e., quantify the increased revenue received by remediation of deficiencies. This example is not limited to estimating revenue savings as measured by federal patient satisfaction targets, but may also be used to estimate revenue savings by reaching other, internal hospital targets. In Report Generator 2, identified deficiencies and estimates of potential revenue savings are documented in various reports for the hospitals. What separates this process from simple patient registries and dashboards is that the calculator may determine which issues warrant the most immediate and future attention, and which have the most potential for improving revenue.

The above steps are intended to be repeated iteratively, in whole or in part, as hospitals develop interventions for improving the childbirth experience and use the above steps to guide them toward satisfaction score improvement.

The inclusion of specific operations and the order of operations shown in the flow diagrams are provided for illustrative purposes only and, in accordance with other embodiments, steps may be removed, reordered, modified, or performed simultaneously.

Although the present disclosure has been described with reference to the example embodiments, those skilled in the art will recognize that various changes and modifications to the described embodiments may be performed, all without departing from the spirit and scope of the present disclosure. Furthermore, those skilled in the various arts will recognize that the present disclosure described herein will suggest solutions to other tasks and adaptations for other applications. It is the applicant's intention to cover by the claims herein, all such uses of the present disclosure, and those changes and modifications which could be made to the example embodiments of the present disclosure herein chosen for the purpose of disclosure, all without departing from the spirit and scope of the present disclosure. Thus, the example embodiments of the present disclosure should be considered in all respects as illustrative and not restrictive, with the spirit and scope of the present disclosure being indicated by the appended claims, and their equivalents. Further, those skilled in the art would appreciate that one or more features according to one more embodiments of the present disclosure may be combined with one or more other features according to one or more other embodiments of the present disclosure without departing from the spirit and scope of the present disclosure.

What is claimed is:

1. An analytical data computing system, comprising:
a processor; and
a memory coupled to the processor, the memory storing instructions which, when executed by the processor, cause the processor to:
receive, from an electronic device, antepartum patient data corresponding to a patient from among a plurality of patients;
compare the antepartum patient data with pre-stored aggregated antepartum patient data;
generate risk factor data based on the comparison of the antepartum patient data with the pre-stored aggregated antepartum patient data;
transmit, to the electronic device, a signal for displaying information corresponding to the risk factor data;
receive, from the electronic device, postpartum patient data corresponding to the patient;
aggregate the postpartum patient data corresponding to the patient with pre-stored aggregated postpartum patient data corresponding to one or more of the plurality of patients to generate updated aggregated postpartum patient data;
calculate, based on the updated aggregated postpartum patient data, one or more performance metric values corresponding to a healthcare provider; and
in response to the one or more performance metric values being below a threshold value, automatically transmit a notification signal to a healthcare provider electronic device, the notification signal comprising an alert message regarding the one or more performance metric values.

2. The analytical data computing system of claim 1, wherein the instructions further cause the processor to identify, based on the antepartum patient data and the pre-stored aggregated postpartum patient data, one or more mitigating factors for the risk factor data.

3. The analytical data computing system of claim 2, wherein the signal for displaying information corresponding to the risk factor data comprises information regarding the one or more mitigating factors.

4. The analytical data computing system of claim 1, wherein the instructions further cause the processor to identify, based on the updated aggregated postpartum data and the one or more performance metric values being below the threshold value, one or more mitigating factors for adjusting the one or more performance metric values.

5. The analytical data computing system of claim 4, wherein the notification signal comprises information regarding the one or more mitigating factors.

6. The analytical data computing system of claim 1, wherein the instructions further cause the processor to:
receive one or more electronic medical records corresponding to the patient; and
automatically generate the risk factor data based on the comparison of the antepartum patient data with the pre-stored aggregated antepartum patient data and the one or more electronic medical records.

7. The analytical data computing system of claim 1, wherein the instructions further cause the processor to calculate, based on the one or more performance metric values, a potential change in a reimbursement amount for a corresponding change in the one or more performance metric values.

8. A method for controlling a data management system, the method comprising:
receiving, by a processor, from an electronic device, antepartum patient data corresponding to a patient from among a plurality of patients;
comparing, by the processor, the antepartum patient data with pre-stored aggregated antepartum patient data;
generating, by the processor, risk factor data based on the comparison of the antepartum patient data with the pre-stored aggregated antepartum patient data;
transmitting, by the processor, to the electronic device, a signal for displaying information corresponding to the risk factor data;
receiving, by the processor, from the electronic device, postpartum patient data corresponding to the patient;
aggregating, by the processor, the postpartum patient data corresponding to the patient with pre-stored aggregated postpartum patient data corresponding to one or more of the plurality of patients to generate updated aggregated postpartum patient data;
calculating, by the processor, based on the updated aggregated postpartum patient data, one or more performance metric values corresponding to a healthcare provider; and
in response to the one or more performance metric values being below a threshold value, automatically transmitting, by the processor, a notification signal to a healthcare provider electronic device, the notification signal comprising an alert message regarding the one or more performance metric values.

9. The method of claim 8, further comprising identifying, by the processor, based on the antepartum patient data and the pre-stored aggregated postpartum patient data, one or more mitigating factors for the risk factor data.

10. The method of claim 9, wherein the signal for displaying information corresponding to the risk factor data comprises information regarding the one or more mitigating factors.

11. The method of claim 8, further comprising identifying, by the processor, based on the updated aggregated postpartum data and the one or more performance metric values being below the threshold value, one or more mitigating factors for adjusting the one or more performance metric values.

12. The method of claim 11, wherein the notification signal comprises information regarding the one or more mitigating factors.

13. The method of claim 8, further comprising:
receiving, by the processor, one or more electronic medical records corresponding to the patient; and
automatically generating, by the processor, the risk factor data based on the comparison of the antepartum patient data with the pre-stored aggregated antepartum patient data and the one or more electronic medical records.

14. The method of claim 8, further comprising:
calculating, by the processor, based on the one or more performance metric values, a potential change in a reimbursement amount for a corresponding change in the one or more performance metric values.

15. An analytical data computing system, comprising:
a processor; and
a memory coupled to the processor, the memory storing instructions which, when executed by the processor, cause the processor to:
receive, from an electronic device, antepartum patient data corresponding to a first patient from among a plurality of patients;
compare the antepartum patient data with pre-stored aggregated antepartum patient data;
generate risk factor data based on the comparison of the antepartum patient data with the pre-stored aggregated antepartum patient data; and
transmit, to the electronic device, a signal for displaying information corresponding to the risk factor data.

16. The analytical data computing system of claim 15, wherein the instructions further cause the processor to:
receive, from the electronic device, postpartum patient data corresponding to the first patient;
aggregate the postpartum patient data corresponding to the first patient with pre-stored aggregated postpartum patient data corresponding to one or more of the plurality of patients to generate updated aggregated postpartum patient data;
calculate, based on the updated aggregated postpartum patient data, one or more performance metric values corresponding to a healthcare provider; and
in response to the one or more performance metric values being below a threshold value, automatically transmit a notification signal to a healthcare provider electronic device, the notification signal comprising an alert message regarding the one or more performance metric values.

17. The analytical data computing system of claim 16, wherein the instructions further cause the processor to identify, based on the updated aggregated postpartum data and the one or more performance metric values being below the threshold value, one or more mitigating factors for adjusting the one or more performance metric values.

18. The analytical data computing system of claim 17, wherein the notification signal comprises information regarding the one or more mitigating factors.

19. The analytical data computing system of claim 15, wherein the instructions further cause the processor to identify, based on the antepartum patient data and the pre-stored aggregated postpartum patient data, one or more mitigating factors for the risk factor data.

20. The analytical data computing system of claim 19, wherein the signal for displaying information corresponding to the risk factor data comprises information regarding the one or more mitigating factors.
